(19) United States
(12) Patent Application Publication
(10) Pub. No.: US 2025/0191241 A1
(43) Pub. Date: Jun. 12, 2025

(54) METHOD FOR REPRESENTING AT LEAST ONE MEDICAL INSTRUMENT IN A HOLLOW ORGAN, DATA PROCESSING APPARATUS, IMAGING SYSTEM, AND COMPUTER PROGRAM PRODUCT

(71) Applicant: Siemens Healthineers AG, Forchheim (DE)

(72) Inventors: Philipp Roser, Erlangen (DE); Felix Denzinger, Herzogenaurach (DE); Marcus Pfister, Bubenreuth (DE)

(21) Appl. No.: 18/967,254

(22) Filed: Dec. 3, 2024

(30) Foreign Application Priority Data
Dec. 6, 2023 (DE) 10 2023 212 280.4

(51) Int. Cl. G06T 11/00; A61B 34/10; G06T 7/00; G16H 30/40

(52) U.S. Cl. CPC G06T 11/00; A61B 34/10; G06T 7/0012; G16H 30/40; A61B 2034/102; A61B 2034/105; G06T 2207/10116; G06T 2207/30101; G06T 2210/41

(57) ABSTRACT

A method for representing at least one medical instrument in a hollow organ is disclosed. In the method, instrument parameters relating to mechanical properties of the at least one medical instrument are obtained, hollow organ parameters relating to mechanical properties of the hollow organ are obtained, a model of the at least one medical instrument in the hollow organ is generated dependent upon the obtained instrument parameters and the obtained hollow organ parameters, at least one image of the at least one medical instrument in the hollow organ is obtained, and a representation of the at least one medical instrument in the hollow organ is generated dependent upon the at least one obtained image and the generated model.

The present patent document claims the benefit of German Patent Application No. 10 2023 212 280.4, filed Dec. 6, 2023, which is hereby incorporated by reference in its entirety.

### TECHNICAL FIELD

The present disclosure relates to a method for representing at least one medical instrument in a hollow organ. The disclosure further relates to a data processing apparatus for carrying out a method of this type, an imaging system with a data processing apparatus of this type, and a corresponding computer program product.

### BACKGROUND

During interventions on hollow organs, in particular vascular interventions, (e.g., minimal invasive vessel interventions), treatments (e.g., placing vascular supports, also referred to as stents), or diagnoses, (e.g., the detection of stenoses), are carried out by way of medical instruments introduced into the body. Such medical instruments may be the aforementioned stents or vascular catheters, in particular microcatheters, and such. The navigation into the individual vessel branches or the placement of stents takes place by way of the rotation and advancing of a guide wire or a catheter at the insertion point, e.g., in the groin. The guide wire may also be regarded as a medical instrument.

Such interventions may be carried out, for example, with visual monitoring by imaging methods, for example, under X-ray monitoring with angiography systems.

Therein, a plurality of X-ray images may be recorded, and a temporally averaged image may be presented. As a consequence of movement arising, for example, through breathing or a heartbeat, a blurred image of the medical instrument may result. It is also possible that the medical instrument and, in particular, its contour is poorly discernible on a single X-ray image, for example, since a contrast level is too low.

From U.S. Pat. No. 9,082,158 B2, a method for real time stent improvement in a live 2D fluoroscopic scene is known. For this, a movement-compensated stent enhancement image is generated from a first set of images in a fluoroscopic imaging sequence in that individual images of the image sequence are summed in a weighted manner. On the basis of the movement-compensated stent enhancement image, a weighting field is generated. For each new image in the fluoroscopic image sequence that is received, the stent is improved in the new image in that the new image is assembled with the movement-compensated stent enhancement image by using the weighting field.

### SUMMARY AND DESCRIPTION

It is an object of the present disclosure, in the context of imaging, to improve the representation of a medical instrument in a hollow organ. The scope of the present disclosure is defined solely by the appended claims and is not affected to any degree by the statements within this summary. The present embodiments may obviate one or more of the drawbacks or limitations in the related art.

The disclosure is based upon the concept, for improved representation of at least one medical instrument of generating a model of the at least one medical instrument in the hollow organ that takes into account mechanical properties of both the at least one medical instrument and also of the hollow organ.

An aspect of the disclosure relates to a method for representing a medical instrument in a hollow organ. Instrument parameters relating to mechanical properties of the at least one medical instrument are obtained. Hollow organ parameters relating to mechanical properties of the hollow organ are obtained. A model of the at least one medical instrument in the hollow organ is generated dependent upon the obtained instrument parameters and the obtained hollow organ parameters. At least one image of the at least one medical instrument in the hollow organ is obtained. A representation, e.g., a visual representation, of the at least one medical instrument in the hollow organ is generated dependent upon the at least one obtained image and the generated model and is, in particular, displayed.

In particular, the method is an imaging method or part of one such. An image of the at least one medical instrument in the hollow organ is an image that maps the hollow organ and the at least one instrument in the hollow organ, for example, an, in particular, two-dimensional X-ray projection image or a computed tomography (CT) reconstruction or a magnetic resonance image (MRI). Thus, the method may also be an X-ray imaging method.

In different embodiments of the method, the method may be entirely computer-implemented. If not otherwise stated, all the acts of such a computer-implemented method may be carried out by a data processing apparatus that has at least one computing unit. In particular, the at least one computing unit is configured or adapted for carrying out the acts of the computer-implemented method. For this purpose, the at least one computing unit may store, for example, a computer program that includes commands that, on execution by the at least one computing unit, cause the at least one computing unit to carry out the computer-implemented method.

From each embodiment of the computer-implemented method, there follows a corresponding embodiment of the method that is not purely computer-implemented in that corresponding method acts for generating the at least one image by an imaging modality, for example, an X-ray-based imaging modality, is included.

The at least one image is generated, for example, by an imaging modality while the at least one medical instrument is being introduced into the hollow organ and/or once the at least one medical instrument has been introduced into the hollow organ and/or the at least one medical instrument is moved within the hollow organ. The introduction of the at least one medical instrument into the hollow organ or the movement of the at least one medical instrument is not part of the method. The generation of the at least one image may be part of the method in certain embodiments and, in other embodiments, may take place before the execution of the method.

In certain embodiments, the at least one image includes a plurality of images.

The hollow organ may be at least one vessel, in particular a blood vessel, for example, a vascular tree with a main vessel and one or more vascular branches.

The at least one medical instrument may include a vascular prosthesis, in particular, a stent and/or a guide wire or a catheter, in particular a vascular catheter. If the at least one medical instrument includes more than one instrument, the mechanical instrument parameters may characterize mechanical properties of individual instruments. In addition, in this case, the mechanical instrument parameters may optionally be characterized by an interaction between the different instruments if they are connected or coupled to one another.

The mechanical instrument parameters may include a geometrical form and/or a diameter and/or a length and/or a stiffness and/or a surface lubricity of the corresponding medical instrument. It is possible that the at least one medical instrument has X-ray-visible markings. Then, the mechanical instrument parameters may also include spacings and/or positions of the markings relative to one another. If the at least one medical instrument includes a balloon catheter, the markings may be configured as balloon markers. The mechanical instrument parameters may be determined before the execution of the method. The determination of the mechanical instrument parameters, in particular, by measuring the corresponding medical instrument may, in certain embodiments, also be part of the method.

The mechanical hollow organ parameters may include a stiffness and/or an elasticity, in particular a bulk modulus, and/or a geometrical form, in particular, a diameter or a progression of the diameter along the longitudinal direction of the hollow organ, of the hollow organ. The mechanical vessel properties may also vary locally, for example, along the longitudinal direction of the main vessel.

The hollow organ parameters may be established by a prior CT image generated, for example, before the intervention. In particular, for this purpose, a prior CT image of a hollow organ region of the hollow organ into which the at least one medical instrument is to be introduced, is obtained, in particular generated, and is provided to the computing unit. From the prior CT image, the hollow organ parameters may be determined particularly precisely and reliably.

The mechanical hollow organ parameters are not necessarily exclusively defined by the hollow organ itself. For example, it is also possible that the tissue surrounding or adjoining the hollow organ, such as for instance internal organs, bones, muscle tissue, fat tissue and suchlike, influence the mechanical hollow organ parameters.

It is possible that the model is generated dependent upon model boundary conditions. For example, the model boundary conditions are generated dependent upon the mechanical instrument parameters and the mechanical hollow organ parameters.

The model optionally includes a mapping and/or an outer contour of the at least one medical instrument for different possible positions of the at least one medical instrument in the hollow organ for specific hollow organ parameters and instrument parameters. Alternatively, the model optionally includes a mapping and/or an outer contour of the at least one medical instrument for a specified, in particular obtained, position of the at least one medical instrument in the hollow organ for specific hollow organ parameters and instrument parameters.

The representation concerns, for example, a visual representation of a display unit, for example, on a screen and/or the representation includes representation data from which such a representation may be generated.

Taking account of the hollow organ parameters and the instrument parameters, the method enables the at least one medical instrument in the hollow organ to be represented better, in particular more readily discernible by an observer.

In one embodiment, as the representation, the at least one image is overlaid with an artificial representation of the at least one medical instrument. The artificial representation is generated dependent upon the model that is generated. The artificial representation may be designated an overlay.

This embodiment enables an observer to recognize particularly easily where the stent is situated.

In an embodiment, the at least one image includes at least two images.

In an embodiment, an image region of the at least two obtained images is selected. For this purpose, for example, a plurality of pixels of the at least two images are selected.

The image region may be selected in each image of the at least two images. In particular, the selected image region maps the same content in each of the at least two images. The at least two images may be different from one another and, in particular, the selected image region shows related or identical image content, e.g., at a different site in the respective image.

For example, the markings are detected in each of the at least two images. The image region may be selected dependent upon positions of the detected markings. For example, for this purpose, at least parts of a method may be carried out as in the document U.S. Pat. No. 9,082,158 B2 mentioned in the introduction.

This is not necessarily a two-dimensional region. It is also possible that only a one pixel-width sequence of pixels is selected as the image region. In certain examples, only outer contours of the at least one medical instrument are selected as the image region. Dependent upon the model, a deformation field is generated for the selected image region. The representation is generated dependent upon the deformation field.

For example, the selected region is deformed in accordance with the deformation field. In particular, the deformed region is displayed in place of the original region.

Optionally, a pixel strength that may also be identified as a gray-scale value, in particular, of pixels from the selected region, is increased. Thereby, the outer contour of the at least one medical instrument may be represented, for example, with improved contrast. A pixel information item for increasing the pixel strength may be part of the deformation field or is provided in addition to the deformation field.

It is possible, for example, that from the model, overlay information that is used, in particular, for generating the artificial representation and, in particular, has an outer contour of the at least one medical instrument at a particular position in the hollow organ, is generated. Optionally, the overlay information corresponds to the model. Alternatively, the overlay information corresponds to the model, taking into account a specific position of the at least one medical instrument in the hollow organ, in particular, if the model has the position as an input parameter. The deformation field may be determined dependent upon the overlay information.

In one embodiment, the deformation field for the image region defines how the image region, or its pixels are rotated when the representation is generated and/or how the size of the image region is changed.

By this process, the at least two images may be configured to the overlay information and, in particular, to one another.

It is possible that, in order to generate the deformation field, a first image of the at least two images is compared with the overlay information. The deformation field may define how the first of the at least two images is rotated and/or displaced in order to have a high, in particular the highest, level of matching with the overlay information.

The at least two images may be represented overlaid following the deformation. This may be designated, in particular, a resultant representation.

Alternatively, or additionally, it is possible that the first of the at least two images is compared with at least one second of the at least two images. For example, the deformation field defines that the outer contour of the at least one medical instrument is represented in the resultant representation at a position that lies between the respective positions of the outer contours of the at least two images, in particular a mean value thereof.

In an embodiment, during the generation of the deformation field, deformation field boundary conditions are taken into account.

It is thereby prevented, in particular a probability is reduced, that the image region changed by the deformation field represents impossible or unlikely positions and/or orientations of the at least one medical instrument.

The deformation field boundary conditions may include that the at least one medical instrument is situated within the hollow organ and/or that the markings of the at least one medical instrument are consistently arranged relative to the at least one medical instrument and/or that a guide wire is situated within the at least one medical instrument.

In an embodiment, in order to generate the model, a finite element method (FEM) simulation is carried out.

Thus, the model is determined particularly precisely for the obtained hollow organ parameters and the obtained instrument parameters. Thereby, the representation of the at least one medical instrument is improved, in particular, more precise.

For the FEM simulation, for example, a corresponding simulation tool is used, for example, Simscale (see https://www.simscale.com/de/), or Simcenter Femap software (see https://plm.sw.siemens.com/de-DE/simcenter/mechanical-simulation/femap/).

The expression FEM simulation may also include extensions such as, for instance, the method of the mixed finite elements.

In an embodiment, allocation data is obtained. The allocation data allocates an outer contour of the at least one instrument in the hollow organ to specified combinations of instrument parameters and hollow organ parameters. The model is generated dependent upon the allocation data.

Therefrom arises the advantage that the model is generated more rapidly as compared with a model that is generated dependent upon a simulation.

In particular, in the allocation data, different sets of instrument parameters and hollow organ parameters are allocated, not or not only the obtained instrument parameters and hollow organ parameters.

The combination of the obtained hollow organ parameters and instrument parameters may not be contained within the allocation data. It is possible that it is checked which combination of instrument parameters and hollow organ parameters come closest to the obtained instrument parameters and hollow organ parameters. The allocated outer contour, in particular, the allocated outer contours for different positions of the at least one medical instrument in the hollow organ may be generated for this closest combination, in particular selected as a model.

In an embodiment, the allocation data is interpolated, dependent upon the obtained instrument parameters and the obtained hollow organ parameters. The model is generated dependent upon the interpolated allocation data.

By this procedure, the model is generated with high accuracy and rapidly.

In this embodiment, dependent upon the obtained instrument parameters and hollow organ parameters, at least two closest combinations present in the allocation data may be determined. In particular, in this embodiment, the outer contours allocated to the at least two combinations, in particular, the allocated outer contours for different positions of the at least one medical instrument in the hollow organ, are interpolated. In this way, the model may be generated as an interpolated contour that, in particular, is either determined for a particular position of the at least one instrument in the hollow organ or has the position as an input parameter of the model.

The outer contour or the outer contours may be interpolated by a polynomial function or as a spline.

In an embodiment, a position to be observed in the hollow organ is obtained. Dependent upon the obtained position that is to be observed, the representation of the at least one instrument is generated. The position to be observed in the hollow organ may be designated, in particular, a vessel portion.

As previously described, the vessel portion, which may also be designated a vessel position, may be an input parameter of the model. The model may be provided before an execution of an intervention in which the at least one medical instrument is introduced into the hollow organ. In certain examples, in the execution, the current position of the at least one instrument in the hollow organ may be obtained and, dependent upon the obtained position, by the model, for example, the overlay information may be generated.

In an embodiment, the mechanical instrument parameters include wire parameters regarding mechanical properties of a guide wire for guiding the at least one instrument in the hollow organ. The model is generated dependent upon the wire parameters.

By this process, an improved model may be generated.

The at least one medical instrument may include the guide wire. Alternatively, the at least one medical instrument does not include the guide wire. Nevertheless, the model may be improved by way of taking account of the wire parameters since, by way of the wire parameters, the behavior of the at least one medical instrument, for example a stent, may be influenced. The wire parameters may describe how the wire is coupled to the at least one instrument.

In an embodiment, the obtained wire parameters define or include a geometrical form and/or a diameter and/or a length and/or a stiffness and/or an elasticity.

In an embodiment, the at least one medical instrument includes a stent or a vascular catheter.

The method is particularly advantageous for these medical instruments since the instruments remain for a long time from a number of hours up to many years in the hollow organ. It is therefore decisive for a correct positioning, in particular, for an effectiveness of the stent. For the correct positioning, a correct representation of the at least one medical instrument is needed. This is improved by way of the method.

In an embodiment, the obtained instrument parameters define or include a geometrical form and/or a diameter and/or a length and/or a stiffness and/or an elasticity and/or a surface lubricity of the at least one instrument.

The stiffness may be a tensile stiffness or a compressive stiffness or a flexural stiffness or a torsional stiffness. The surface lubricity is, in particular, a lubricity of an outer surface of the respective stent or vascular implant. The lubricity is given, for example, by an inverse of a coefficient of friction.

The diameter may be an internal diameter or an external diameter. The diameter may also be different at different sites of the respective stent or vascular implant.

In an embodiment, the at least one image is at least one X-ray image, in particular an X-ray projection image.

According to a further aspect, a method for supporting an intervention on a hollow organ is provided. Therein, at least one medical instrument is introduced into a hollow organ of a person. A method for representing the at least one medical instrument in the hollow organ is carried out.

Therein, the hollow organ parameters and the instrument parameters are obtained, for example, before the introduction of the at least one medical instrument into the hollow organ. The model may also be generated before the introduction of the at least one medical instrument into the hollow organ. The at least one image of the at least one medical instrument is obtained, in particular, after the introduction of the at least one medical instrument into the hollow organ. In particular, the representation of the at least one medical instrument is generated after the introduction of the at least one medical instrument into the hollow organ.

According to a further aspect, a data processing apparatus is provided. The data processing apparatus has at least one computing unit that is configured to carry out a method for representing at least one medical instrument in a hollow organ.

A computing unit may be understood, in particular, to be a data processing device that includes a processing circuit. The computing unit may thus process, in particular, data to carry out computation operations. This also covers, where relevant, operations to carry out indicated access operations on a data structure, for example, a look-up table (LUT).

The computing unit may include one or more computers, one or more microcontrollers, and/or one or more integrated circuits, for example, one or more application-specific integrated circuits (ASICs), one or more field-programmable gate arrays (FPGA) and/or one or more system on a chip (SoC) units. The computing unit may also include one or more processors, for example, one or more microprocessors, one or more central processing units (CPUs), one or more graphics processing units (GPUs) and/or one or more signal processors, in particular, one or more digital signal processors (DSPs). The computing unit may also include a physical or a virtual network of computers or others of the aforementioned units.

In different embodiments, the computing unit includes one or more hardware and/or software interfaces and/or one or more storage units.

A storage unit may be configured as a volatile data store, for example, as a dynamic random access memory (DRAM) or as a static random access memory (SRAM) or as a non-volatile data store, for example, a read-only memory (ROM) as a programmable read-only memory (PROM) as an erasable programmable read-only memory (EPROM) as an electrically erasable programmable read-only memory (EEPROM) as a flash memory or flash-EEPROM, as a ferroelectric random access memory (FRAM) as a magnetoresistive random access memory (MRAM) or as a phase-change random access memory (PCRAM).

According to a further aspect, an imaging system is provided that has a data processing apparatus as well as an imaging modality configured to generate the at least one image.

According to at least one embodiment, the imaging modality is an X-ray imaging modality with an X-ray source and an X-ray detector.

According to at least one embodiment, the X-ray imaging modality is configured as a C-arm device.

According to a further aspect, a computer program with commands is provided. If the commands are executed by way of a data processing apparatus, in particular, a data processing apparatus, the commands cause the data processing apparatus to carry out a method.

The commands may be present as program code. The program code may be provided as binary code or assembler and/or as source code of a programming language, for example, C and/or as a program script, for example, Python.

According to a further aspect, a computer-readable storage medium is also provided that stores a computer program.

The computer program and the computer-readable storage medium are each computer program products with the commands.

Further features and feature combinations of the disclosure are disclosed in the drawings and their description as well as in the claims. In particular, further embodiments do not necessarily include all the features of one of the claims. Further embodiments of may have features or combinations of features that are not named in the claims.

The disclosure is now described in greater detail by reference to specific embodiments and the associated schematic drawings. In the figures, the same or functionally identical elements may have been provided with the same reference signs. The description of the same or functionally identical elements is, where relevant, not necessarily be repeated in relation to different drawings.

### BRIEF DESCRIPTION OF THE DRAWINGS

FIG. 1 depicts a schematic illustration of an embodiment of an imaging system.

FIG. 2 depicts a schematic flow diagram of an embodiment of a method for representing at least one medical instrument in a hollow organ.

FIG. 3 depicts a schematic illustration of an example of a representation of at least one medical instrument in a hollow organ.

### DETAILED DESCRIPTION

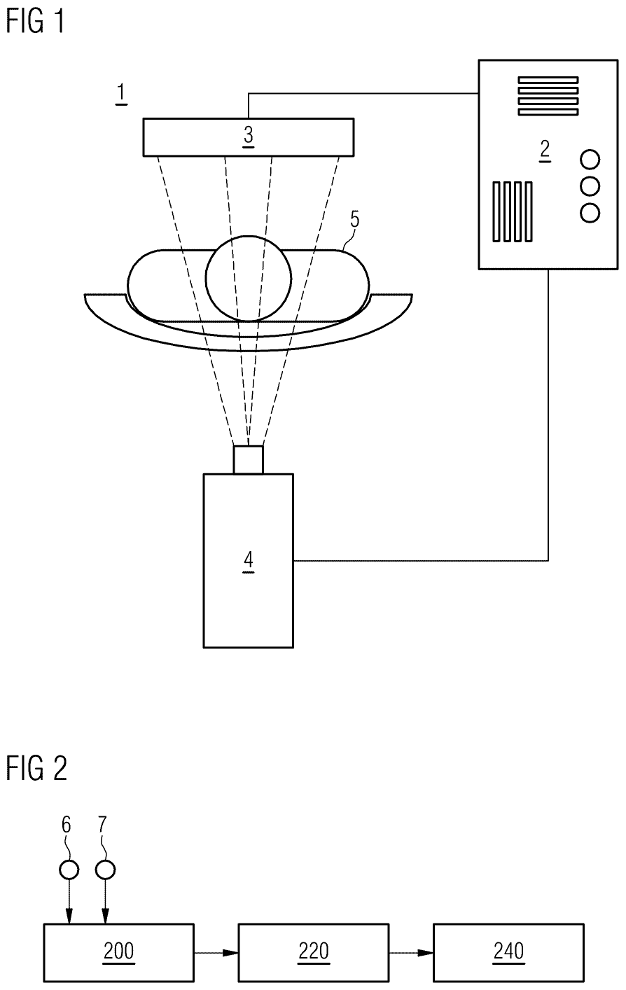

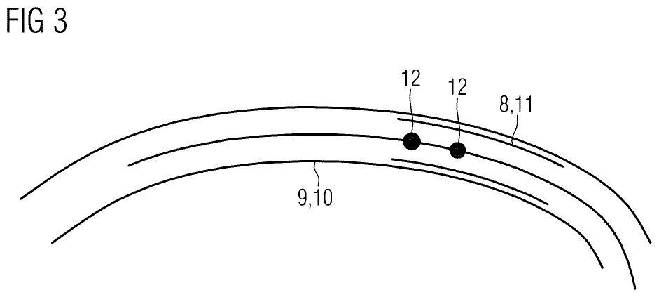

FIG. 1 shows a schematic illustration of an embodiment of an imaging system 1. The imaging system 1 has an imaging modality 3, 4 that is represented in the present case, without restriction of the generality, as an X-ray imaging modality with an X-ray detector 3 and an X-ray source 4. The imaging system 1 has a data processing apparatus with at least one computing unit 2 configured to carry out a method for representing at least one medical instrument 8 (FIG. 3) in a hollow organ (FIG. 3), in particular in a hollow organ 9 of a patient 5. FIG. 2 shows a schematic flow diagram of an embodiment of such a method.

The at least one computing unit 2 receives the mechanical instrument parameters 6 of the at least one medical instrument 8 and the mechanical hollow organ parameters 7 of the hollow organ 9. The mechanical instrument parameters 6 may be determined, in particular, by way of a measurement of at the at least one medical instrument 8.

In particular, the computing unit 2 receives additional wire parameters that relate to mechanical properties of a guide wire for guiding the at least one instrument 8 in the hollow organ 9. The mechanical instrument parameters 6 include, for example, the wire parameters. By way of example, the at least one medical instrument 8 includes, for example, a stent 11 and the guide wire.

In act 200 of the method, the at least one computing unit 2 generates a model of the at least one medical instrument 8 in the hollow organ 9 on the basis of the mechanical instrument parameters 6, which may include the wire parameters, and the mechanical hollow organ parameters 7. The mechanical instrument parameters 6 include, for example, a geometrical form and/or a diameter and/or a length and/or a stiffness and/or an elasticity and/or a surface lubricity of the at least one medical instrument 8. The mechanical hollow organ parameters 7 may include a stiffness and/or an elasticity of the hollow organ 9. The mechanical hollow organ parameters 7 may also, in particular, vary locally, for example, along the longitudinal direction of a main vessel.

The model may be generated, for example, by a simulation based upon the finite element method, wherein the simulation may be based, for example, upon preoperative planning data or a preoperative CT reconstruction. Alternatively, or additionally, the model may be generated dependent upon allocation data that allocates a contour of the at least one instrument 8 in the hollow organ 9 to predetermined combinations of instrument parameters 6 and hollow organ parameters 7. In particular, for this purpose, the at least one computing unit 2 carries out an interpolation of the allocated contours for the obtained instrument parameters 6 and the obtained hollow organ parameters 7.

In act 220 of the method, the at least one computing unit 2 receives at least one image, in particular at least two images that have been generated by the imaging modality 3, 4. The at least one image shows a vessel portion 10 of a vascular tree of the patient 5. The at least one image shows the at least one medical instrument 8 arranged in the vessel portion 10. The at least one medical instrument 8 may include a stent 11 and possibly the guide wire, as shown schematically in FIG. 3.

In act 240 of the method, a representation of the at least one medical instrument 8 in the hollow organ 9 is generated dependent upon the at least one obtained image and the generated model and is, in particular, displayed.

In an embodiment, the at least one computing unit 2 receives a current position of the at least one medical instrument 8. For example, the at least one computing unit 2 generates overlay information by the model and dependent upon the current position that, in particular, is an input parameter of the model.

The stent 11 shown in FIG. 3 has, in particular, X-ray opaque markings 12. For example, the markings 12 are arranged spaced axially from one another on the stent 11. In particular, by way of the at least one computing unit 2, the current position of the stent 11 may be acquired dependent upon the markings 12. For example, marker positions of the markings 12 are, in particular, further input parameters of the model.

In an embodiment, the overlay information is displayed as an artificial representation at the site in the at least one image at which the at least one medical instrument 8 is, in particular probably, situated. By way of example, the at least one image includes at least two images. Then, in particular, by way of the at least one computing unit 2 from the at least two images, a resultant image is generated. In particular, the artificial representation is displayed in the resultant image.

In an embodiment, dependent upon the overlay information, a deformation field is generated. The at least one image is rotated and/or changed in its size, possibly dependent upon the deformation field. In particular, the rotated and/or changed image is displayed as a representation.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for representing at least one medical instrument in a hollow organ, the method comprising:
obtaining instrument parameters relating to mechanical properties of the at least one medical instrument;
obtaining hollow organ parameters relating to mechanical properties of the hollow organ;
generating a model of the at least one medical instrument in the hollow organ dependent upon the instrument parameters and the hollow organ parameters;
obtaining at least one image of the at least one medical instrument in the hollow organ; and
generating a representation of the at least one medical instrument in the hollow organ dependent upon the at least one image and the model.

2. The method of claim 1, wherein the representation comprises the at least one image overlaid with an artificial representation of the at least one medical instrument, and
wherein the artificial representation is generated dependent upon the model.

3. The method of claim 1, wherein the at least one image comprises at least two images,
wherein an image region of the at least two images is selected,
wherein, dependent upon the model, a deformation field is generated for the selected image region, and
wherein the representation is generated dependent upon the deformation field.

4. The method of claim 3, wherein the deformation field for the image region defines how the image region is rotated when the representation is generated and/or how a size of the image region is changed.

5. The method of claim 1, wherein the generating of the model comprises carrying out a finite element simulation.

6. The method of claim 1, wherein allocation data that allocates a contour of the at least one medical instrument in the hollow organ to predetermined combinations of the instrument parameters and the hollow organ parameters is obtained, and
wherein the model is generated dependent upon the allocation data.

7. The method of claim 6, wherein the allocation data is interpolated dependent upon the instrument parameters and the hollow organ parameters, and
wherein the model is generated dependent upon the interpolated allocation data.

8. The method of claim 1, wherein a position to be observed in the hollow organ is obtained, and
wherein the representation of the at least one medical instrument is generated dependent upon the obtained position to be observed.

9. The method of claim 1, wherein the at least one mechanical instrument parameters comprise wire parameters regarding mechanical properties of a guide wire for guiding the at least one medical instrument in the hollow organ, and
wherein the model is generated dependent upon the wire parameters.

10. The method of claim 1, wherein the at least one medical instrument comprises a stent or a vascular catheter.

11. The method of claim 1, wherein the instrument parameters of the at least one medical instrument comprise a geometrical form, a diameter, a length, a stiffness, an elasticity, a surface lubricity, or a combination thereof.

12. The method of claim 1, wherein the at least one image is at least one X-ray image.

13. A data processing apparatus comprising:
at least one computing unit configured to:
obtain instrument parameters relating to mechanical properties of at least one medical instrument;
obtain hollow organ parameters relating to mechanical properties of a hollow organ;
generate a model of the at least one medical instrument in the hollow organ dependent upon the instrument parameters and the hollow organ parameters;
obtain at least one image of the at least one medical instrument in the hollow organ; and
generate a representation of the at least one medical instrument in the hollow organ dependent upon the at least one image and the model.

14. An imaging system comprising:
a data processing apparatus having at least one computing unit configured to:
obtain instrument parameters relating to mechanical properties of at least one medical instrument;
obtain hollow organ parameters relating to mechanical properties of a hollow organ;
generate a model of the at least one medical instrument in the hollow organ dependent upon the instrument parameters and the hollow organ parameters;
obtain at least one image of the at least one medical instrument in the hollow organ; and
generate a representation of the at least one medical instrument in the hollow organ dependent upon the at least one image and the model; and
an imaging modality configured to generate the at least one image of the at least one medical instrument in the hollow organ.
